(19) United States
(12) Patent Application Publication
(10) Pub. No.: US 2011/0208022 A1
(43) Pub. Date: Aug. 25, 2011

(54) DEVICE AND METHODS FOR SAMPLING PROSTATE FLUID

(75) Inventors: Michael K. Brawer, Mercer Island, WA (US); Isa Rizk, San Diego, CA (US); John Fulkerson, Ranch Santa Margarita, CA (US); Stephen Sosnowski, Vista, CA (US); George Wallace, Coto de Caza, CA (US)

(73) Assignee: Intersect Partners ,LLC a Limited Liability Corporation

(21) Appl. No.: 13/119,135

(22) PCT Filed: Sep. 14, 2009
(86) PCT No.: PCT/US2009/056861

§ 371 Date: May 10, 2011

Related U.S. Application Data
(60) Provisional application No. 61/097,457, filed on Sep. 16, 2008.

(51) Int. Cl. A61B 5/145; A61B 5/15; A61M 25/10

(52) U.S. Cl. 600/309; 600/581; 604/101.03

(57) ABSTRACT

Disclosed are devices and methods to improve sampling of prostate secretions for purposes of diagnoses, staging, assessment of malignant potential, prognosis.

### CROSS-REFERENCE TO RELATED APPLICATIONS

This application is a U.S. National Phase of International Application No. PCT/US2009/056861, filed Sep. 14, 2009, designating the U.S. and published in English on Mar. 25, 2010 as WO 2010/033467, which claims the benefit of U.S. Provisional Application No. 61/097,457 filed Sep. 16, 2008, the entirety of which is hereby incorporated by reference herein and made a part of the present specification.

### BACKGROUND OF THE INVENTION

The present invention is generally related to devices for collection of prostate secretions, that include formed elements for the purpose of diagnosing different conditions of the prostate and surrounding tissues in a patient.

The structure that compose the male genital system can give rise to disease because of malformation, inflammation and neoplasms. Along with the testes and penis, the prostate, one of the anatomical subdivisions of the male genitalia, can present with these disorders in a very selective fashion. Prostatic disease thus, can appear as an inflammatory lesion (prostatitis), benign hyperplasia and carcinoma. Each of these presentations can have an effect on the tissues, organs or systems neighboring the prostate, or affect the whole human body as when prostatic cancer metastasizes. The glandular epithelial tissue produces prostatic fluid. The smooth muscles contract during sexual climax and squeeze the prostatic fluid into the urethra as the sperm passes through the ejaculatory ducts and urethra. Prostatic fluid secreted by the prostate gland provides nutrition for ejaculated sperm increasing their mobility and improves the sperm chances for survival after ejaculation by making the environment in the vaginal canal less acidic and viscous.

A clinical apparent inflammation of the prostate, prostatitis, may be acute or chronic, this classification based on a combination of clinical features, microscopic analysis of urine, and at times further bacterial culture of fractionated urine. The organisms causing infection of the bladder, urethra may cause concomitant infection of the prostate. Acute prostatitis thus, is characterized by acute neutrophilic inflammatory infiltrates, congestion and stromal edema. In chronic prostatitis there are non-specific histologic features and often include variable amounts of lymphoid infiltrate, evidence of glandular injury and acute inflammatory changes. The clinical manifestation of both include dysuria, urinary frequency, lower back pain and poorly localized suprapubic or pelvic pain. Chronic bacterial prostatitis is one of the most important causes of urinary tract infection in men.

Benign prostatic hyperplasia (BPH) occurs generally in men over 50 years of age; by age 60, approximately 50% of men have histologic evidence of BPH and 15% have significant lower urinary symptoms. The etiology of BPH appears to be related to hormonal changes, such as serum testosterone levels that slowly but significantly decrease with advancing age; however levels of estrogenic steroids are not decreased equally. According to this theory the prostate enlarges because of increased estrogenic effects. It is likely that the secretion of intermediate peptide growth factors plays a part in the development of BPH.

It is important to realize that the relationship between anatomical prostatic enlargement (BPH), the symptoms of prostatitis and urodynamic evidence of bladder outflow obstruction (BOO) is complex. Anatomically the effects can be: 1) the prostatic urethra is lengthened, sometimes twice its normal length, but it is not narrowed anatomically. The normal posterior curve may be so exaggerated that it requires a curved catheter to negotiate it. When only one lateral lobe is enlarged, distortion of the prostatic urethra occurs. 2) BPH causes BOO, the musculature of the bladder hypertrophies to overcome the obstruction. Significant BPH is associated with increased blood flow and the resultant veins at the base of the bladder are apt to cause hematuria. Also BOO will lead to impaired bladder emptying, the bladder decompensates with the development of a large volume of residual urine, urinary infection and calculi are prone to develop. Generally the assessment of BPH is initially made by abdominal or rectal examination. The uncertain benefit of early detection and radical treatment of prostate cancer, may aid ascertain BPH with the use of a prostate specific antigen (PSA) test.

Carcinoma of the prostate is the most common non cutaneous malignant tumor in men. Prostate cancer is a major cause of death among men in Western countries. In 2007 in the United States there were 218,890 new cases diagnosed and 27,050 men succumbed to this disease. The current protocol for detection of this cancer involves testing for prostate-specific antigen (PSA) levels. If PSA levels are found to be high (generally ≧4 ng/ml), a tissue biopsy is performed most often under ultrasound guidance transrectally with a spring loaded biopsy device. PSA testing is limited by the fact that it lacks both sensitivity and more importantly specificity and it does not distinguish between prostate cancer and benign prostate hyperplasia. As a result, many men either are not identified as having the disease or because of false positive tests are subjected to the invasive tissue biopsies when they do not, have the disease. A much more specific and less invasive diagnostic test is needed for early detection of this disease.

U.S. Pat. No. 5,919,163 issued on Jul. 6, 1999, the entire contents of which are incorporated herein by reference, discloses a double balloon catheter and methods of use, which includes a first fixed balloon on the cranial end of the catheter, a second slidable balloon positioned around the catheter and spaced from the first fixed balloon with structure for enabling the second balloon to slide along the plastic tube.

U.S. Pat. No. 5,662,608 issued on Sep. 2, 1999, the entire contents of which are incorporated herein by reference, discloses a low profile catheter comprising a flexible elongate tubular member having a guide wire lumen and a first and a second balloon wherein the first and second inflatable balloons are offset from each other longitudinally of the flexible elongate tubular member or wherein the first and second inflatable balloons are mounted eccentrically in opposite directions from each other to provide a combined profile of larger diameter.

U.S. Pat. No. 7,060,051 issued on Jun. 13, 2006, the entire contents of which are incorporated herein by reference, discloses a multi-balloon catheter for occluding two ends of a portion of a blood vessel and treating the occluded portion of the blood vessel, wherein the balloons are capable of being inflated and deflated within the blood vessel, and when the balloons are inflated, form a substantially fluid-tight, closed space between the first and second balloons.

What is clinically needed is a sampling device, such as a catheter or needle, that is capable of occluding a portion of the urethra of a patient and extracting fluid from within the portion for collection of prostate secretions for the purpose of diagnosing different conditions of the prostate and surrounding tissues in a patient

### SUMMARY OF THE INVENTION

It is one aspect of the present invention to improve collection of prostate secretions and/or fluid and/or cells and/or proteins and/or DNA and/or RNA and/or carbohydrates for purposes of diagnoses, staging, assessment of malignant potential, prognosis, and others.

Current approach (PCA3) is to analyze urine for prostate secretions, fluid, cells, proteins, DNA, RNA, and/or carbohydrates for the purposes of diagnoses, staging, assessment of malignant potential, and prognosis. The disadvantage of this method is that the marker sample is diluted within the urine, making the specificity of the test less sensitive. Concentrating the test sample will improve sensitivity and overall accuracy of the test.

Some aspects of the invention provide a balloon catheter for occluding two ends of a portion of a urethra and sampling fluid within the occluded portion comprising: a first inflatable balloon for occluding a first end of the portion of the urethra; a second inflatable balloon for occluding a second end of the portion and for anchoring the catheter at the second end, wherein the second balloon is configured with a cylindrical exterior surface for frictionally anchoring the second balloon against a urethra wall; and a catheter segment connectively placed between the first and second balloons; wherein the catheter segment is capable of collecting the fluid from the occluded portion for sampling the fluid within the occluded portion and when the balloons are inflated, form a substantially fluid-tight, closed space between the first and second balloons.

In one embodiment, the catheter segment of the balloon catheter of the present invention comprises an inner lumen, and a catheter wall and wherein at least one port extends through the wall to the exterior of the catheter segment, the port permitting aspiration of the fluid to pass through from the exterior to the inner lumen of the catheter segment.

In one embodiment, the balloon catheter of the present invention comprises a handle, an inner lumen and an elongate catheter sheath having a sheath distal end and a sheath proximal end, wherein the sheath distal end is connected to a proximal end of the catheter segment and the sheath proximal end is connected to the handle with fluid communication inside the inner lumen.

In one embodiment, the handle of the balloon catheter of the present invention comprises at least three fluid communication ports to external sources, the handle being characterized with a substantially flat configuration and all fluid communicating ports are on the same flat plane.

In a preferred embodiment, the balloon catheter of the present invention comprises an external suction means for collecting the fluid from the occluded portion for fluid sampling.

In one embodiment, the balloon catheter of the present invention comprises a third-balloon disposed between the first and second balloons and a catheter segment connectively placed between the first and third balloons and a catheter segment connectively placed between the second and third balloons.

In one embodiment, the balloon catheter of the present invention comprises low amplitude vibration energy that is configured to dislodge various prostate analytes from prostate epithelium or stroma and thus, allow prostate analytes to be present in greater concentration in prostatic ducts or prostatic urethra for sampling.

In one embodiment, the catheter segment of the balloon catheter of the present invention comprises an expandable segment, the expandable segment comprising at least a center tubing with a lumen for providing inflation fluid to the first inflation balloon, wherein the expandable segment may comprise one or more fluid tubing with ports for fluid sampling. In one embodiment, the expandable segment comprises one or more expandable elements, the expandable elements being configured like an expandable basket, wherein the expandable elements are deployed to expand and press the urethra wall so to effect greater secretion of prostatic fluid.

Some aspects of the invention provide a balloon catheter for occluding two ends of a portion of a urethra and treating the occluded portion comprising: a first inflatable balloon for occluding a first end of the portion of the urethra; a second inflatable balloon for occluding a second end of the portion and for anchoring the catheter at the second end, wherein the second balloon is configured with a cylindrical exterior surface for frictionally anchoring the second balloon against a urethra wall; and a catheter segment connectively placed between the first and second balloons; wherein the catheter segment is capable of delivering a biologically active agent to the occluded portion for treating the occluded portion and wherein the balloons are capable of being inflated and deflated within the urethra, and when the balloons are inflated, form a substantially fluid-tight, closed space between the first and second balloons.

In one embodiment, the catheter segment of the balloon catheter of the present invention comprises an inner lumen, and a catheter wall and wherein at least one port extends through the wall to the exterior of the catheter segment, the port permitting the biologically active agent to pass through from the inner lumen to the exterior of the catheter segment.

In one embodiment, the biologically active agent of the balloon catheter of the present invention is selected from the group consisting of chemotherapy, cytotoxics, cytostatics, antibiotics, growth factors, growth inhibitors, radiation, hormonal agents, and vitamins.

In one embodiment, the biologically active agent of the balloon catheter of the present invention is selected from the group consisting of cells, proteins, DNA, RNA and carbohydrates.

Some aspects of the invention provide a method for concentrating an extractable test compound in a bodily conduit of a patient, comprising: (a) providing a balloon catheter, wherein the balloon catheter comprises a first inflatable balloon for occluding a first end of the conduit; a second inflatable balloon for occluding a second end of the conduit and for anchoring the catheter at the second end, wherein the second balloon is configured with a cylindrical exterior surface for frictionally anchoring the second balloon against a conduit wall; and a catheter segment connectively placed between the first and second balloons; wherein the catheter segment is capable of concentrating the test compound by flushing with a fluid; (b) deploying the balloon catheter to the conduit; (c) inflating both balloons to form a substantially fluid-tight, closed space between the first and second balloons; and (d) flushing the closed space with the fluid via the catheter segment, thereby concentrating the test compound inside the closed space. In one embodiment, the fluid is infused from a fluid source outside of the patient. In another embodiment, the flushing step is repeated at least one more time. In still another embodiment, the extractable test compound is prostatic fluid. In a further embodiment, the bodily conduit is not limited to a urethra or a blood vessel. Further, the bodily conduit may be selected from group consisting of veins, esophagus, duodenum, biliary tract, pancreas, small bowel, colon, ureter, fallopian tube, uterus, vas deferens, trachea, bronchus, bronchioles, mammary ducts, Fallopian tubes, and Testis.

### BRIEF DESCRIPTION OF THE DRAWINGS

Additional features of the present invention will become more apparent and the invention itself will be best understood from the following Detailed Description of Exemplary Embodiments, when read with reference to the accompanying drawings.

FIG. 1 shows a vertical view of the anatomy of a prostate gland and its relative location with respect to adjacent organs in a male patient.

FIG. 2 shows a horizontal sectional view of the anatomy of a prostatic urethra and its relative location with respect to adjacent organs in a male patient.

FIG. 3 shows a preferred embodiment of a catheter with an occluding balloon and an occluding/anchoring balloon: (A) when balloons are at a deflated stage; and (B) when balloons are at an inflated stage.

FIG. 4 shows a detailed outer configuration of the catheter of FIG. 3: (A) a side-view; and (B) a top-view.

FIG. 5 shows (A) an outer configuration of the catheter of FIG. 3, (B) a cross-sectional view of the portion of the handle, section I-I, and (C) a cross-sectional view of the portion of the elongate catheter segment between the balloons, section II-II.

FIG. 6 shows one embodiment of catheter balloons with an elongate radially expandable segment between the balloons; (A) when balloons are at a deflated stage; (B) when balloons are at an inflated stage; and (C) an expanded section for detailed viewing, section III.

### DETAILED DESCRIPTION OF THE PREFERRED EMBODIMENT

The preferred embodiments of the present invention described below relate particularly to a device system and methods for collection of prostate secretions, fluid, cells, proteins, DNA, RNA and/or carbohydrates for purposes of diagnoses, staging, assessment of malignant potential, prognosis, and others. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

### Anatomy and Physiology of Prostate

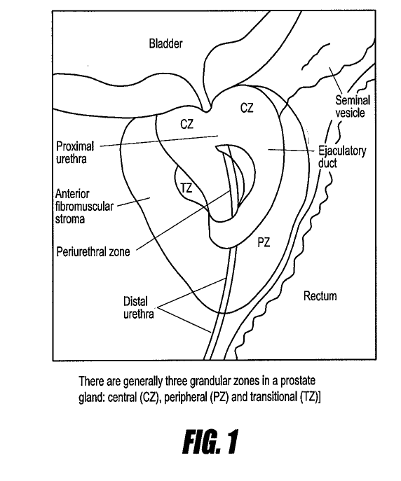

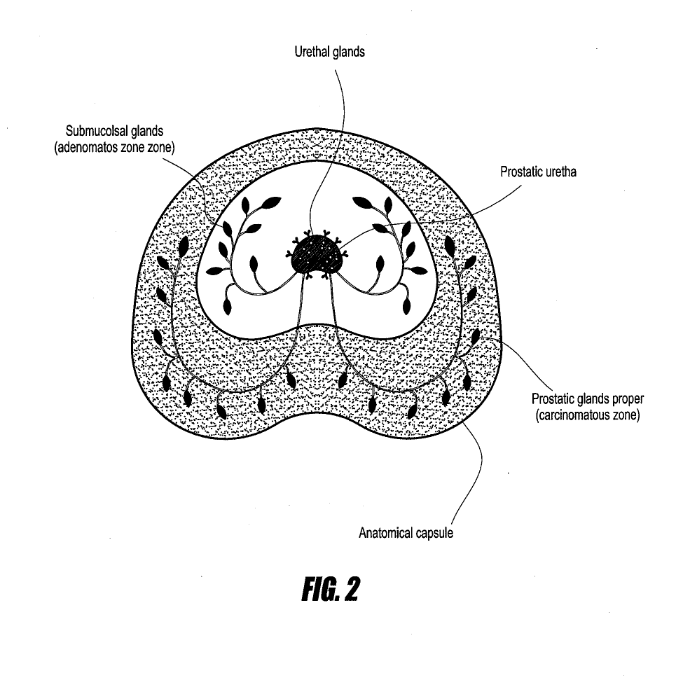

The prostate is a walnut-sized gland that forms part of the male reproductive system. The gland consists of several lobes, or regions, enclosed by a dense fibrous capsule. It is located between the bladder and the rectum and wraps around the urethra, the conduit that carries urine out from the bladder through the penis. There are generally three glandular zones in a prostate gland (FIG. 1): central (CZ), peripheral (PZ) and transitional (TZ). The transitional zone is located right behind the place where the seminal vesicles are merging with urethra. This transitional zone tends to be predisposed to benign enlargement. The prostate gland is generally composed of smooth muscles and glandular epithelial tissue. A transverse section of the prostate in the region of the peripheral zone and the adenomatous zone that composes the central and transitional zones are shown in FIG. 2, and this is the area from which most prostate cancers arise.

BPH (benign prostatic hyperplasia) typically affects the submucuous group of glands in the transitional zone, forming a nodular enlargement. Eventually, this overgrowth compresses the PZ glands into a false capsule and causes the appearance of the typical “lateral” lobes. When BPH affects the subcervical central zone glands, a “middle” lobe develops which projects up into the bladder within the internal sphincter. Sometimes both lateral lobes project into the bladder, so that when viewed from within, the sides and back of the internal urinary meatus are surrounded by an intravesical prostatic collar.

If PSA levels are found to be high (≧4 ng/ml), a tissue biopsy is generally performed by means of Turkel's needle.

Some aspects of the invention relate to a device system for enhancing the collection of prostate secretions for purposes of diagnoses, staging, assessment of malignant potential, and prognosis. One aspect is to enhance concentration in a specimen of informative material or signal (for example, a biomarker) and thereby improve sensitivity of a diagnostic test. This device would accomplish this object by increasing access to fluids containing the informative material within the prostatic ducts in lieu of urine analysis. A “biomarker” is a biologically occurring characteristic or molecule that may be measured in a biological sample and evaluated as an indicator of normal biologic or pathogenic processes; of biological or pathogenic status; as an index of the risk or the progression of disease; as a measure of exposure to an environmental chemical or factor; and/or of pharmacological response to a therapeutic intervention. Examples of biomarkers include, but are not limited to, hormones, DNA, RNA, protein, peptide, carbohydrate, lipid and steroids.

In one exemplary embodiment, it is disclosed a multi-port catheter with an occlusion/anchoring device in the bladder neck, and an occlusion device at the distal end of the prostatic urethra to isolate the prostatic urethra. Side ports in continuity with a separate access port are utilized to irrigate the sampling region (i.e., prostatic urethra) in a barbottage manner to maximize the concentration of the informative analyte in the sample. In one embodiment, low amplitude vibration energy is implemented in order to increase concentration of the collected sample. In another embodiment, other device characteristics are summarized below. A catheter would generally be the one with a size about 4-20 F, preferably 7-12 F, in diameter.

The catheter is characterized with blind placement with a proximal occluding/anchoring device and a distal occluding device, wherein the occluding device may be made of silicone, polyurethane, or other expandable polymeric material. In one alternate embodiment, the catheter has a distal occluding/anchoring device and a proximal occluding device. The distance between the distal and proximal occluding devices is generally about 1-10 cm, preferably 3-5 cm, wherein the distance may be adjustable via sliding/multiple shaft design. The catheter is sized and configured with controlled injection/aspiration with open ducts. The catheter device may be supplemented with vacuum suction, agitation or means for enhancing aspiration, such as turbulent flow, lavage cycling, mechanical, thermal, vibration energy or low amplitude vibration energy, sonic waves, pulsating rinse mechanism, rotating rinse, heated rinse and so forth.

In one embodiment, low amplitude vibration energy is configured to dislodge various prostate analytes from the prostate epithelium or stroma (benign and malignant including pre-malignant entities) and allow them to be present in greater concentration in the prostatic ducts and prostatic urethra. This vibration energy can be used in specimens obtained in voided urine, prostatic secretions obtained post digital rectal examination or prostatic massage, specimens obtained by catheterization. The vibration device may be incorporated in the prostatic fluid sampling catheter. The vibration device may comprise piezoelectric element, ultrasonic element, electromagnetic element, or mechanical means.

In one preferred embodiment, the device is used to infuse drugs/chemicals to the tissue site for therapeutic, diagnostic or sampling purposes. For example, one may use this device (or similar version with an injecting needle or port) to deliver therapeutics to the prostate or a target tissue site including chemotherapy, cytotoxics, cytostatics, antibiotics, growth factors, growth inhibitors, radiation, hormonal agents, vitamins, etc. Device traits/notes include: device can utilize higher pressures to force open ducts within the prostate; and device can deliver therapeutics to the prostate and keep them there for an extended period of time.

In a further embodiment, a 3rd balloon/device (between an occluding device and a spaced-apart occluding/anchoring device) to apply light pressure on prostate is optional on the catheter device of the present invention. Other features can also be added to the sampling device, such as brush/swab/sponge mechanism, sliding expandable fixture/cuff, retractable spray ports (enabling closer/more direct rinse), proximal funnel for easy capture of sample that could be expanded by proximal balloon, and any combination of the items of above. In an alternate embodiment, the body fluid sample using the current catheter device may be selected from the group consisting of blood, prostate fluid, urine, seminal fluid, and prostate organ fine needle aspirate fluid samples.

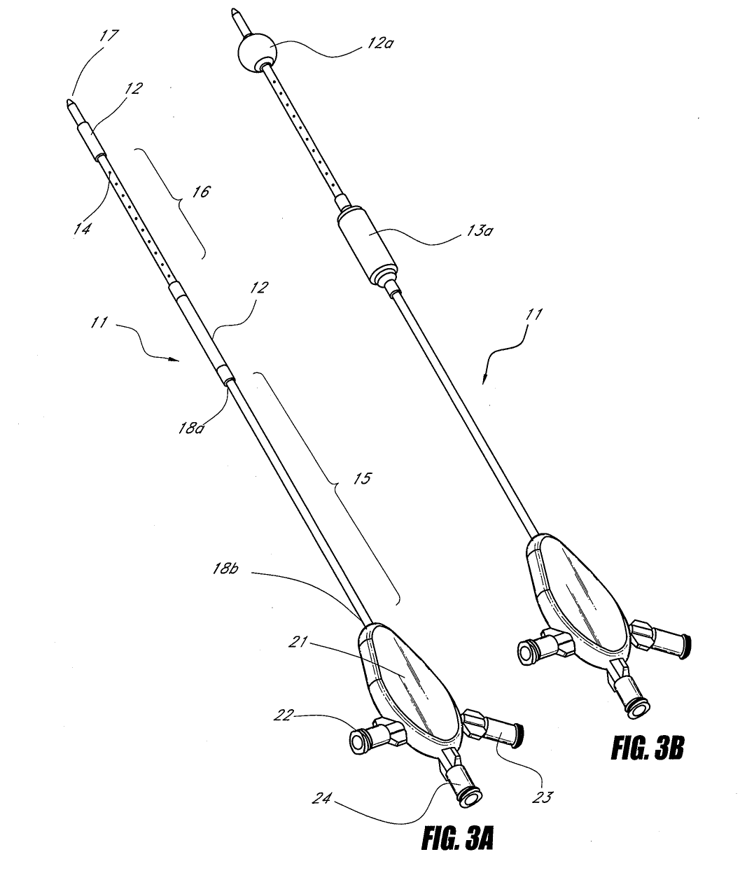

FIG. 3 shows a preferred embodiment of a balloon catheter (11) with an occluding balloon (12) at a distal region that is proximal to the catheter distal end (17) and an occluding/anchoring balloon (13) at a proximal region of the catheter, whereas FIG. 3(A) shows balloons (12, 13) at a deflated stage suitable for delivering the balloon catheter with low profile to the prostatic urethra site and FIG. 3(B) shows balloons (12a, 13a) at an inflated stage to create an isolated portion within the urethra for fluid sampling or fluid infusing when deployed. The balloon catheter has a catheter segment (16) connectively placed between and under the first balloon (12) and the second balloon (13), wherein the catheter segment comprises an inner aspiration lumen (14c), and a catheter wall and wherein at least one port (14) extends through the wall to the exterior of the catheter segment (16), the port permitting aspiration of the fluid to pass through from the exterior of the catheter segment to the inner lumen of the catheter segment for sampling fluid to an outside collecting instrument. In an alternate embodiment, the catheter segment comprises an inner flushing lumen (14b), and a catheter wall and wherein at least one port extends through the wall to the exterior of the catheter segment (16), the port permitting infusing of the fluid to pass through the inner lumen of the catheter segment to the exterior of the catheter segment.

In one embodiment, the balloon catheter comprises an ergonomic or flat-face handle (21), an inner lumen (10) and an elongate catheter sheath (15) having a sheath distal end (18a) and a sheath proximal end (18b), wherein the sheath distal end is connected to a proximal end of the catheter segment (16) and the sheath proximal end (18b) is connected to the handle (21) with fluid communication inside the inner lumen. In one embodiment, the inner lumen (10) comprises at least a flushing lumen (14b), an aspiration lumen (14c), a distal balloon inflation lumen (12b), and a proximal balloon inflation lumen (not shown). In another embodiment, the handle (21) may comprise at least three fluid communication ports connecting to external fluid sources, the handle being characterized with a substantially flat configuration and all fluid communication ports are on the same flat plane. The handle having a substantially flat configuration with all fluid communication ports on the same flat plane is particularly applicable for sampling prostate fluid with ease.

Inflation fluid or saline may be delivered from the first fluid communicating port (22) through the distal balloon inflation lumen (12b) inside the catheter sheath (15) and the catheter segment (16) to the distal balloon (12). Similarly, inflation fluid or saline may be delivered from the second fluid communicating port (23) through the proximal balloon inflation lumen (not shown) inside the catheter sheath (15) to the proximal balloon (13). In one embodiment, a third fluid communicating port (24) is provided to aspirate fluid via the aspiration lumen (14c) from the isolated portion of the urethra between the two occluding balloons. Optionally, a fourth fluid communicating port (not shown) is provided to infuse fluid via the flushing lumen (14b) to the isolated portion of the urethra between the two occluding balloons.

Some aspects of the invention relate to a balloon catheter for occluding two ends of a portion of a urethra and sampling fluid within the occluded portion comprising: a first inflatable balloon for occluding a first end of the portion of the urethra; a second inflatable balloon for occluding a second end of the portion and for anchoring the catheter at the second end, wherein the second balloon is configured with a cylindrical exterior surface for frictionally anchoring the second balloon against a urethra wall; and a catheter segment connectively placed between the first and second balloons; wherein the catheter segment is capable of collecting the fluid from the occluded portion and when the balloons are inflated, form a substantially fluid-tight, closed space between the first and second balloons.

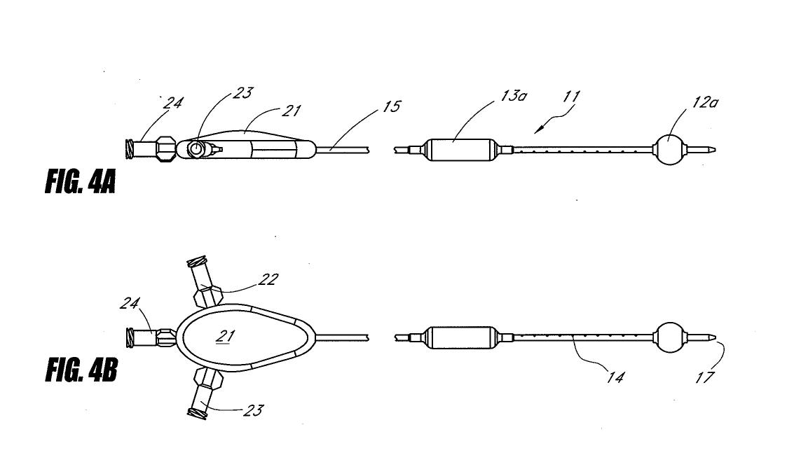

FIG. 4 shows a detailed outer configuration of the catheter of FIG. 3: (A) a side-view; and (B) a top-view with inflated balloons (12a, 13a). As shown, some first aspiration ports (14) are on one side of the catheter segment (16) and some second ports are located at certain angles apart from the first aspiration ports, for example 90° or 180° apart.

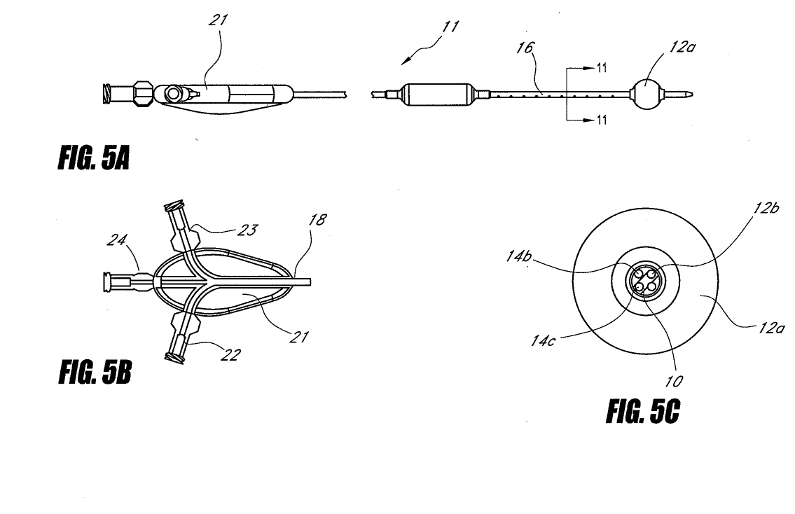

FIG. 5 shows (A) an outer configuration of the catheter of FIG. 3; (B) a cross-sectional view of the portion of the handle (21); and (C) a cross-sectional view of the portion of the elongate catheter segment (16) between the balloons (12a, 13a).

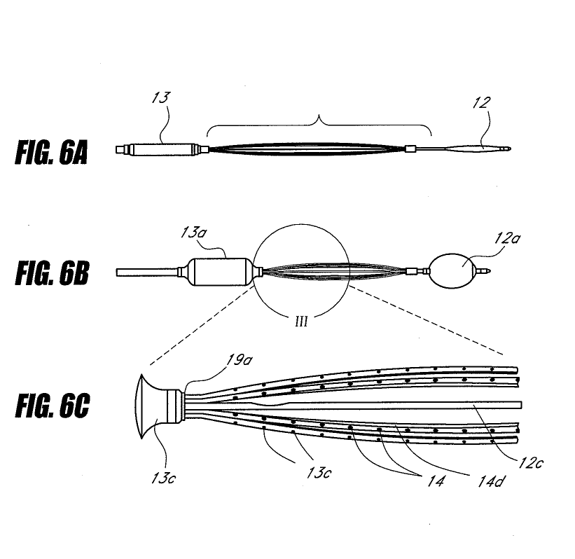

FIG. 6 shows one embodiment of catheter balloons with an elongate radially expandable segment (19) between the balloons; (A) when balloons (12, 13) are at a deflated stage; (B) when balloons (12a, 13a) are at an inflated stage; and (C) an expanded section for detailed viewing, section III. In one embodiment, the distal section (13c) of the proximal balloon (13) is connected to the proximal end (19a) of the expandable segment (19). In another embodiment, the expandable segment comprises at least a center tubing (12c) with a lumen for providing inflation fluid to the distal balloon (12), one or more fluid tubing (14d) with ports (14), and one or more expandable elements (19b). The expandable elements can be configured and activated like an expandable basket. In one preferred embodiment, the expandable elements are deployed to expand and press the wall of the portion of the urethra between the occluded balloons so to effect more secretion of prostatic fluid.

### Procedure for Prostatic Fluid Sampling

1. Obtain patient consent.
2. Patient given a P.O. Quinolone 1 hour prior to procedure.
3. Patient voids emptying bladder.
4. Perform Directed Digital Rectal Examination.
5. Insert lubricated IP Prostate Sampling Device into urethra so that distal 5 cm. is within the bladder.
6. Inflate the distal balloon with 5 cc. of saline (Port 1).
7. Pull down the device so distal balloon sits at bladder neck occluding the urethra distally.
8. Inflate distal balloon with 2-4 cc to occlude urethra below the prostatic urethra (Port 2).
9. Keep gentle traction on device to maintain occlusion of bladder neck.
10. Inject 3-5 cc of sample collection fluid in Port 3. Aspirate fluid and repeat flushing (barbottage) up to 20 times.
11. Aspirate fluid and place in specimen container.
12. Remove device.
13. Add buffer solution.
14. Ship to reference laboratory.

From the foregoing, it should now be appreciated that a device system for collection of prostate secretions or fluid (including cells, proteins, DNA, RNA, and carbohydrates) for purposes of diagnoses, staging, assessment of malignant potential, prognosis has been disclosed. While the invention has been described with reference to a specific embodiment, the description is illustrative of the invention and is not to be construed as limiting the invention. Various modifications and applications may occur to those skilled in the art without departing from the true spirit and scope of the invention as described by the appended claims.

What is claimed is:

1. A balloon catheter for occluding two ends of a portion of a urethra and sampling fluid within the occluded portion comprising:
a first inflatable balloon for occluding a first end of said portion of the urethra;
a second inflatable balloon for occluding a second end of said portion and for anchoring said catheter at the second end, wherein the second balloon is configured with a cylindrical exterior surface for frictionally anchoring said second balloon against a urethra wall; and
a catheter segment connectively placed between the first and second balloons;
wherein said catheter segment is capable of collecting the fluid from the occluded portion for sampling the fluid within the occluded portion and when the balloons are inflated, form a substantially fluid-tight, closed space between the first and second balloons.

2. The balloon catheter of claim 1, wherein said catheter segment comprises an inner lumen, and a catheter wall and wherein at least one port extends through the wall to the exterior of the catheter segment, said port permitting aspiration of the fluid to pass through from the exterior to the inner lumen of the catheter segment.

3. The balloon catheter of claim 1 which comprises a handle, an inner lumen and an elongate catheter sheath having a sheath distal end and a sheath proximal end, wherein said sheath distal end is connected to a proximal end of said catheter segment and said sheath proximal end is connected to the handle with fluid communication inside the inner lumen.

4. The balloon catheter of claim 3, wherein the handle comprises at least three fluid communication ports to external sources, the handle being characterized with a substantially flat configuration and all fluid communicating ports are on the same flat plane.

5. The balloon catheter of claim 1 which comprises an external suction means for collecting the fluid from the occluded portion for fluid sampling.

6. The balloon catheter of claim 1 which comprises a third-balloon disposed between the first and second balloons and a catheter segment connectively placed between the first and third balloons and a catheter segment connectively placed between the second and third balloons.

7. The balloon catheter of claim 1 which comprises low amplitude vibration energy that is configured to dislodge various prostate analytes from prostate epithelium or stroma and thus, allow prostate analytes to be present in greater concentration in prostatic ducts or prostatic urethra for sampling.

8. The balloon catheter of claim 1, wherein the catheter segment comprises an expandable segment, the expandable segment comprising at least a center tubing with a lumen for providing inflation fluid to the first inflation balloon.

9. The balloon catheter of claim 1, wherein the expandable segment comprises one or more fluid tubing with ports for fluid sampling.

10. The balloon catheter of claim 1, wherein the expandable segment comprises one or more expandable elements, the expandable elements being configured like an expandable basket, wherein the expandable elements are deployed to expand and press the urethra wall so to effect greater secretion of prostatic fluid.

11. A balloon catheter for occluding two ends of a portion of a urethra and treating the occluded portion comprising:
a first inflatable balloon for occluding a first end of said portion of the urethra;
a second inflatable balloon for occluding a second end of said portion and for anchoring said catheter at the second end, wherein the second balloon is configured with a cylindrical exterior surface for frictionally anchoring said second balloon against a urethra wall; and
a catheter segment connectively placed between the first and second balloons;
wherein said catheter segment is capable of delivering a biologically active agent to the occluded portion for treating the occluded portion and wherein said balloons are capable of being inflated and deflated within the urethra, and when the balloons are inflated, form a substantially fluid-tight, closed space between the first and second balloons.

12. The balloon catheter of claim 11, wherein said catheter segment comprises an inner lumen, and a catheter wall and wherein at least one port extends through the wall to the exterior of the catheter segment, said port permitting the biologically active agent to pass through from the inner lumen to the exterior of the catheter segment.

13. The balloon catheter of claim 11, wherein the biologically active agent is selected from the group consisting of chemotherapy, cytotoxics, cytostatics, antibiotics, growth factors, growth inhibitors, radiation, hormonal agents, and vitamins.

14. The balloon catheter of claim 11, wherein the biologically active agent is selected from the group consisting of cells, proteins, DNA, RNA and carbohydrates.

15. A method for concentrating an extractable test compound in a bodily conduit of a patient, comprising:
(a) providing a balloon catheter, wherein said balloon catheter comprises a first inflatable balloon for occluding a first end of said conduit; a second inflatable balloon for occluding a second end of said conduit and for anchoring said catheter at the second end, wherein the second balloon is configured with a cylindrical exterior surface for frictionally anchoring said second balloon against a conduit wall; and a catheter segment connectively placed between the first and second balloons; wherein said catheter segment is capable of concentrating said test compound by flushing with a fluid;
(b) deploying said balloon catheter to said conduit;
(c) inflating both balloons to form a substantially fluid-tight, closed space between the first and second balloons; and
(d) flushing said closed space with said fluid via the catheter segment, thereby concentrating said test compound inside said closed space.

16. The method of claim 15, wherein said catheter segment comprises an inner lumen, and a catheter wall and wherein at least one port extends through the wall to the exterior of the catheter segment, said port permitting the biologically active agent to pass through from the inner lumen to the exterior of the catheter segment for flushing.

17. The method of claim 16, wherein said catheter segment further comprises second inner lumen, and wherein at least one port extends from said second inner lumen through the wall to the exterior of the catheter segment, said port permitting aspiration of the extractable test compound to pass through from the exterior to the second inner lumen of the catheter segment.

18. The method of claim 15, wherein the extractable test compound is prostatic fluid.

19. The method of claim 15, wherein the bodily conduit is a urethra.

20. The method of claim 15, wherein the bodily conduit is selected from the group consisting of blood vessel, veins, esophagus, duodenum, biliary tract, pancreas, small bowel, colon, ureter, fallopian tube, uterus, vas deferens, trachea, bronchus, and bronchioles.

21. The method of claim 15, wherein the bodily conduit is selected from the group consisting of mammary ducts, Fallopian tubes and the ductus deferens of the testes.

22. The method of claim 15, wherein said fluid is infused from a fluid source outside of the patient.

23. The method of claim 15, wherein the flushing step is repeated at least one more time.
